(19) United States
(12) Patent Application Publication
(10) Pub. No.: US 2006/0025398 A1
(43) Pub. Date: Feb. 2, 2006

(54) Topical antiviral therapeutic and prophylactic treatment of adenoviruses and their associated diseases

(76) Inventor: David Gershon, New York, NY (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 11/097,081

(22) Filed: Mar. 31, 2005

Related U.S. Application Data
(63) Continuation-in-part of application No. 10/883,406, filed on Jun. 30, 2004.
(60) Provisional application No. 60/484,234, filed on Jun. 30, 2003.
(60) Provisional application No. 60/557,544, filed on Mar. 30, 2004.

(30) Foreign Application Priority Data
Jun. 30, 2004 (WO) PCT/US04/21218
Mar. 31, 2005 (WO) PCT/US05/10890

(51) Int. Cl. A61L 9/04; A61K 31/555

(52) U.S. Cl. 514/184; 424/45

(57) ABSTRACT

A method for the therapeutic and prophylactic treatment of adeviruses, More specifically, a method for the therapeutic treatment of adenovirus in a subject by topically administering an antiviral effective amount of CTC-96 to the subject. In addition, a method for the prophylactic treatment against an adenovirus infection in a subject by topically administering a prophylactically anti-adenovirus effective amount of CTC-96 to the subject to minimize the likelihood of the subject veing infected by the adenovirus.

### CROSS REFERENCES TO RELATED APPLICATIONS

This application is a continuation-in-part application of application Ser. No. 10/883,406 filed Jun. 30, 2004, which claims priority of U.S. Provisional Application No. 60/484,234 filed Jun. 30, 2003, the entire disclosure of which are hereby incorporated by reference.

### BACKGROUND OF THE INVENTION

Adenoviruses commonly infect the eye, respiratory and gastrointestinal tracts and can infect other organs such as the liver, urinary bladder, pancreas, central nervous system and others. There are over 50 known serotypes of Human Adenoviruses of which at least 24 have been identified as pathogens. Adenovirus has been shown to persist for months after initial infection in particular in immunosuppressed patients.

TABLE 1
Adenoviruses Serotypes and Disease

| Disease | Major Serotypes* |
|---|---|
| Acute febrile pharyngitis | 1, 2, 3, 5, 6, 7 |
| Acute respiratory disease | 3, 4, 7, 14, 21 |
| Acute hemorrhagic cystitis | 11, 21 |
| Epidemic keratoconjunctivitis | 8, 11, 19, 37 |
| Gastroenteritis | 40, 41 |
| Hepatitis | 1, 2, 5 |
| Meningoencephalitis | 7, 12, 32 |
| Pertussis-like syndrome | 5 |
| Pharyngoconjuctival fever | 3, 7, 14 |
| Pneumonia (children) | 1, 2, 3, 7 |
| Pneumonia (adults - military recruits) | 4, 7 |

*Serotypes in bold with underline have been tested and shown to be sensitive to CTC-96

An example of Adenovirus-Related Disease

Adenoviruses are the most prevalent causes of acute ocular viral disease for which there is no known cure. The actual prevalence and incidence of Epidemic Keratoconjunctivitis (EKC) caused by Adenoviruses in the U.S. and internationally are unknown, because general practitioners and optometrists see most cases and this infection does not have to be reported to any medical authority. EKC is highly contagious and has the tendency to occur in epidemics.

While EKC is a self-limiting disease that generally resolves within 1-3 weeks the patient may remain highly infectious for 10-14 days or more after symptoms develop (I). Symptoms of EKC include conjunctival redness, swelling or redness of the eyelid, discharge from the eye, sticking together of eyelids, pain or discomfort in the eye, photophobia, or a sensation of a foreign body in the eye. In Severe cases, membranous and pseudomembranous conjunctivitis can be seen in one third of cases, which can lead to conjunctival scarring and symblepharon formation (adherence of the bulbar and palpebral conjunctivas) (2; 3). Both membranes and pseudomembranes can occur in EKC with a distinguishing corneal involvement that ranges from diffuse, fine, superficial keratitis to epithelial defects to subepithelial opacities (2; 3). In 20-50% of cases, corneal opacities can persist for weeks to months to several years (I; 3). This phenomenon can decrease visual acuity significantly and cause glare symptoms (2).

There is no specific direct antiviral chemotherapy against Adenoviruses at present. Corticosteroids may be used to limit corneal damage but have the side effects referred to above and also of interfere with viral clearance (3; 4).

### SUMMARY OF THE INVENTION

We have discovered an effective method for the treatment for Human Adenoviruses, and, in particular, Adenovirus-derived keratoconjunctivitis for both therapeutic and prophylactic purposes and respiratory disease. The treatment for adenovirus-derived keratoconjunctivitis, whether it be for therapeutic or prophylactic purposes, can be achieved by topical administration. The treatment for respiratory disease may be by injection or by nasal administration, i.e., by spray or nose drops. As used herein, the expression “therapeutic treatment” means treatment for a subject already having the disease. As used herein, the expression “prophylactic treatment” means treatment for a subject who, while not being infected by the virus, is in a situation wherein they are susceptible to or subject to the possibility of acquiring the disease, e.g., in a household where another resident is already infected with the disease. We have also shown in vitro that CTC-96 is effective against types 1, 2, 3, 4, 5, and 7, attesting to the effectiveness of CTC-96 against the adenovirus derived diseases outlined in Table 1. More particularly, we have discovered that in the eye there is a significant reduction in Adenovirus-derived keratoconjunctivitis disease can be achieved by the by the topical administration of an anti-adenovirus therapeutic or prophylactic effective amount of Compound CTC-96.

As used herein, the word “therapeutic” means use of the inventive method to treat a subject who has already been infected with Adenovirus. As used herein, the word “prophylactic” means use of the inventive method to protect or decrease the likelihood of a subject who may be exposed to Adenovirus from being infected with the virus.
Compound CTC-96 has the structure: 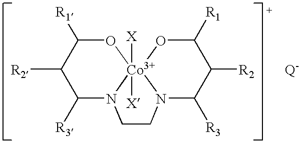
wherein R₁ and R_{1′} are methyl, R₂ and R_{2′} are hydrogen and R₃ and R_{3′} are methyl, and X and X′ are each: 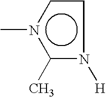
and Q′ is Br⁼.

CTC-96 may be prepared by the method described in the U.S. Pat. No. 5,756,491, the contents of which are hereby incorporated by reference.

Generally, this compound is administered topically in the form of an aqueous solution.

### BRIEF DESCRIPTION OF THE DRAWINGS

FIG. 1 is a graph of Human Adenovirus titers following exposure to CTC-96 prior to cell infection;

FIG. 2 is a graph of virus tiers after exposure of Human Adenovirus infected cells to CTC-96;

FIG. 3 is a graph depicting the effect of treatment of Adenovirus Induced Keratoconjunctivitis with CTC-96; and

FIG. 4 is a graph depicting adenovirus titers after treatment of Adenovirus infected rabbit eyes with CTC-96.

FIG. 5 is a draft of virus titers versus drug concentration.

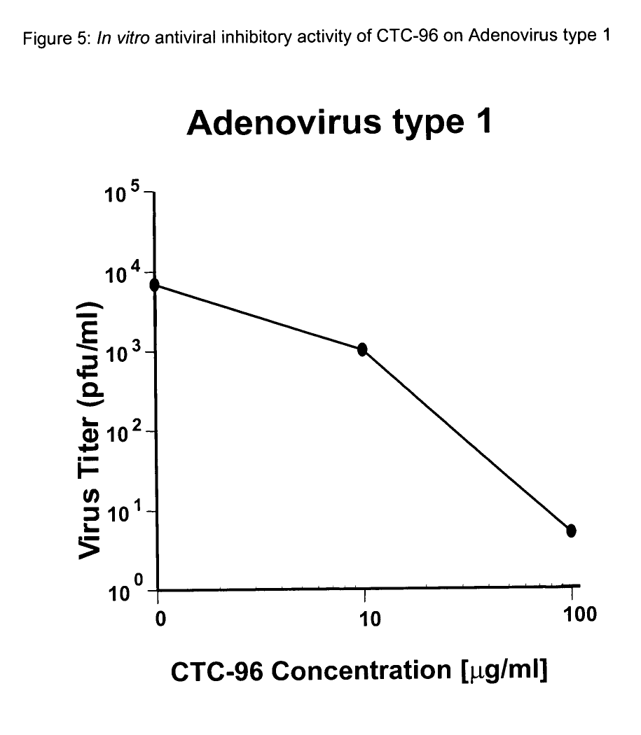

FIG. 6 is a draft of virus titers versus drug concentration.

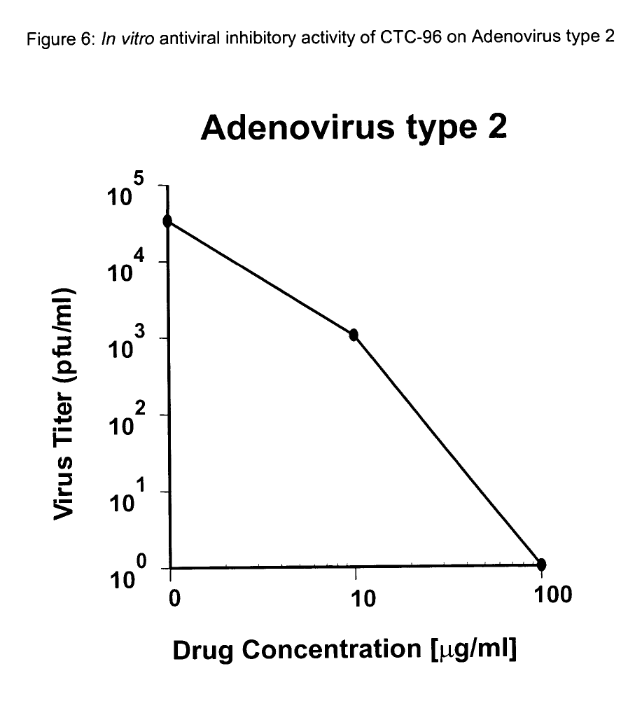

FIG. 7 is a draft of virus titers versus drug concentration.

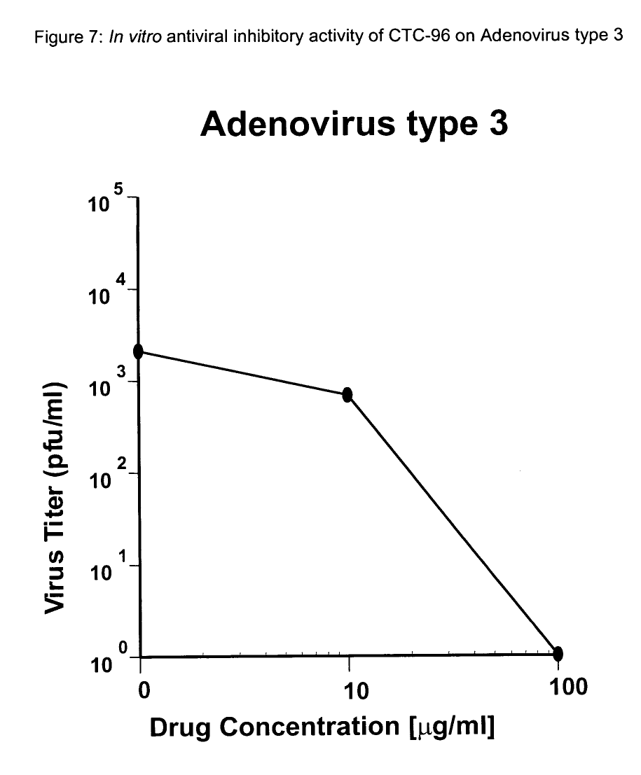

FIG. 8 is a draft of virus titers versus drug concentration.

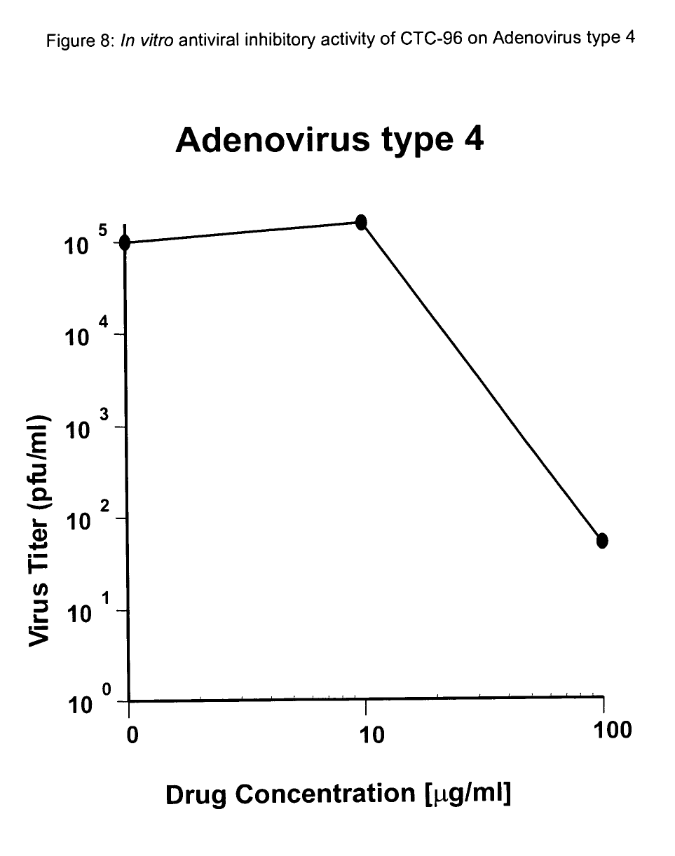

FIG. 9 is a draft of virus titers versus drug concentration.

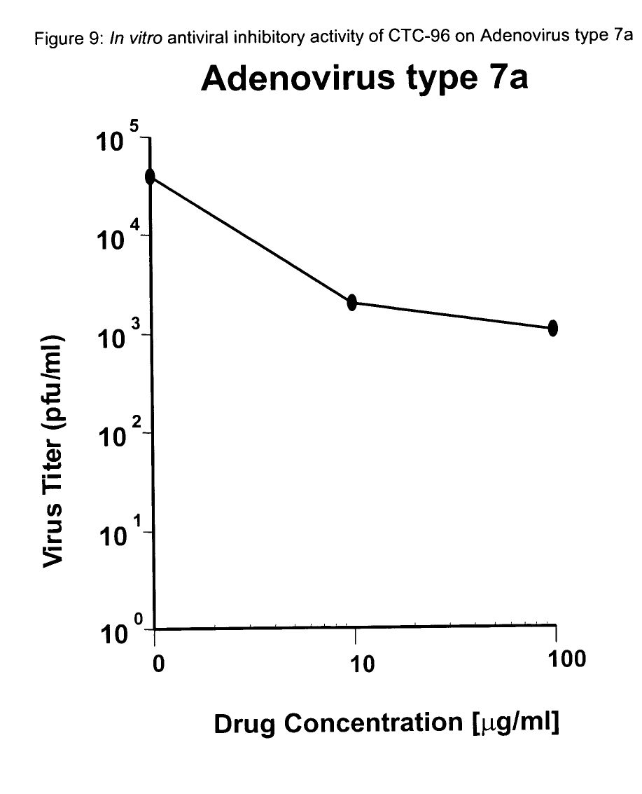

### DETAILED DESCRIPTION OF THE INVENTION

We have demonstrated, by using Adenovirus type 5, that we can reproduce human Adenovirus Infection in rabbit eyes and have shown both excellent antiviral activity and conjunctivitis therapy using CTC-96 which we believe is unique as there is no effective drug against this virus and its pathology in the eye. In addition, we have shown CTC-96 efficacy against Adenovirus types 1, 2, 3, 4, 5, and 7 in HeLa cells in tissue culture. Since these human viruses cannot be grown in animal models, this provides an excellent indication of the effectiveness of CTC-96 against a broad spectrum of Adenovirus types. To determine CTC-96 efficacy against several types of serotypes of adevirus the following procedure was followed:

1. Hela cells were confluent at the time of inoculation.

2. Virus dilutions were prepared from the known titers of the stock viruses (4×10⁵ pfu/ml; 4×10⁴/0.1 ml) of Ad1 Kmetz, Ad2 Wolf, Ad3 Holyfield, Ad4 Harris, Ad7a Joseph, ATCC. This virus inoculation yielded a virus infection with an m.o.i. (multiplicity of infection) of approximately 1.0.

3. 100 □l of each Ad serotype were inoculated onto cultures containing Hela cells.

4. During the adsorption period, Doxovir concentrations of 500, 250, 100, 50, 10, and 0 μg/ml were prepared in culture medium according to the dilution protocol.

5. Virus was adsorbed at 37° C. in a 5% CO₂ water-vapor atmosphere for 1 hour.

6. After adsorption, the virus inocula were removed from all the wells and 2 wells each were overlayed with 1 ml of Doxovir (in tissue culture medium) at concentrations of 500, 250, 100, 50, 10, and 0 μg/ml.

7. The plates were incubated at 37° C. in a 5% CO₂ water-vapor atmosphere for 24 hours.

8. After 24 hours, the plates were washed.

9. Each well was refilled with 1 ml of fresh tissue culture medium without Doxovir.

10. The cells were scraped from the wells.

11. The media and cells were then frozen at −75° C. pending titrations.

12. Titration of duplicate samples were thawed from each Ad serotype, Doxovir concentration and its no drug control.

13. Viral titers were determined at each drug concentration.

CTC-96 has considerable advantages as an anti-viral drug: a) because of its unique mode of action it Is effective against herpes and HIV virus mutants which are resistant to currently used drugs; b) because the drug acts against two different viral targets in herpes virus the development of CTC-96-resistant mutants is deemed to be extremely rare; and c) because CTC-96 has anti-inflammatory properties its use replaces the use of steroids in herpes virus and Adenovirus therapeutics. Steroids modulate the immune response in the areas where they are applied and increase tissue susceptibility to pathogens.

Efficacy Studies

Efficacy of CTC-96 against Adenovirus types 1, 2, 3, 4, 5, and 7 in culture

Anti-adenovirus activity of CTC-96 was evaluated by standard cell culture using HeLa cells, a human cervical carcinoma immortalized cell line (the usual host for laboratory grade adenovirus) and anti-viral plaque-reduction assays. CTC-96 has an inhibitory (prophylactic) effect on growth when virus is exposed to the drug prior to cell infection.

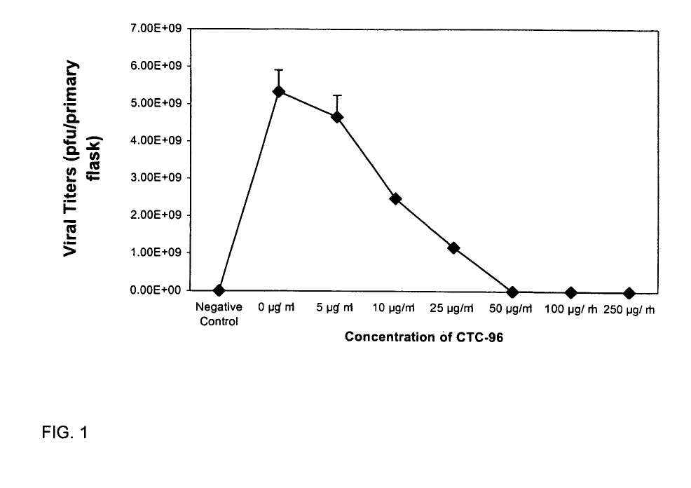

FIG. 1 shows Adenovirus type 5 titers following direct exposure of the virus to CTC-96 prior to HeLa cell infection.

The data graphically depicted in FIG. 1 were obtained as follows: varying concentrations of the CTC-96 were mixed with concentrated Human Adenovirus, [Adenovirus type 5 (Ad5)] and incubated at 37° C. for 60 minutes. Aliquots were then diluted 500 fold into growth medium. Hela cells were exposed to 100 μl of the diluted material to initiate infection. These monolayers were incubated for 24 hours at 37% and 5% CO2 and then washed, scraped, sonicated, centrifuged and the supernatant serially diluted. These serial dilutions were plated onto indicator HeLa cell monolayers and adsorbed for 60 min, aspirated and a methycellulose overlay placed over the cells, which were then incubated for 3 days at 37%. Cultures were counterstained with 1% methylene blue, allowed to dry and the plaques counted. Results are expressed as mean±SD (where error bars are not visible they are contained within data point).

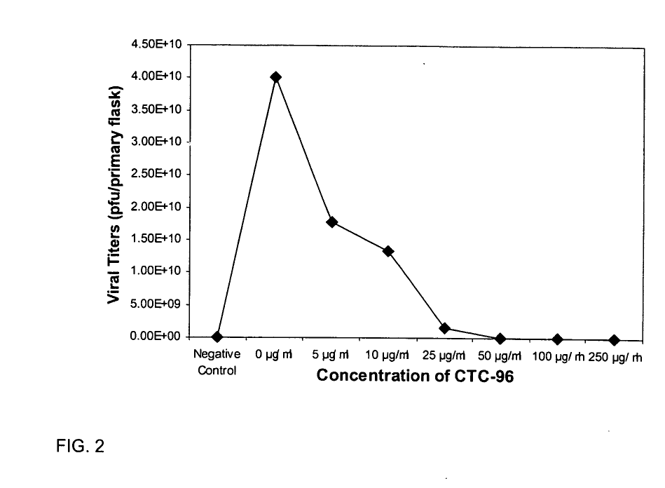

CTC-96 also has a potentially therapeutic effect as can be seen by inhibition of viral growth in Adenovirus infected cells, which are subsequently exposed to the drug. FIG. 2 shows virus titers obtained after exposure of human Adenovirus type 5 (Ad5) infected HeLa cells to CTC-96. These data were obtained as follows: Adenovirus was adsorbed onto HeLa cell monolayers for 60 min at 37%; serial dilutions of CTC-96 were overlaid onto the minelayers. Monolayers were then incubated for 24 hr at 37° C. and 5% CO2. Monolayers were then washed, scraped, sonicated, centrifuged and the supernatant serially diluted. These serial dilutions were plated onto indicator HeLa cell monolayers and adsorbed for 60 min, aspirated and a methylcellulose overlay placed over the cells, which were then incubated for 3 days at 37%. Cultures were counterstained with 1% methylene blue, allowed to dry and the plaques counted. Results am expressed as mean & SD (where error bars are not visible they are contained within data point).

Clinical results and plaque assay viral titers of three CTC-96 treatment/dosing regimens of rabbit eyes infected with Human adenovirus, Adenovirus type 5 (Ad5), were evaluated. On “Day 1” animals were infected with Human Adenovirus Type 5 by the installation of 10⁶ pfu adenovirus according to our protocol of conjunctival and corneal scarification for the induction of Keratoconjunctivitis. Clinical conjunctivitis was observed in all animals by day 8 post-inoculation. Animals were then randomized and the following experimental groups were treated with CTC-96 or placebo in a double blind experiment:
(1) Placebo (diluent alone), 9×/day, for 21 days: (4 rabbits).
(2) CTC-96 50 μg/ml, 9×/day, for 21 days: (4 rabbits).
(3) CTC-96 50 μg/ml, 6×/day, for 21 days: (4 rabbits).
(4) C T W 25 μg/ml, 6×/day, for 21 bays: (4 rabbits).

Clinical disease progression and resolution were evaluated by slit lamp microscopy on days 1, 3, 7, 10, 13, 18, 21, 24, 28 and 31 after initial drug dosing. The intensity of the keratitis was quantified using a clinical grading system (5).

Application of 25 μg/ml and 50 μg/ml prevented progression of disease severity. Application of 50 μg/ml 6 or 9 times a day for 21 days resulted in complete resolution of clinical disease by day 21 while placebo treated animals continued to show symptoms for another 10 days.

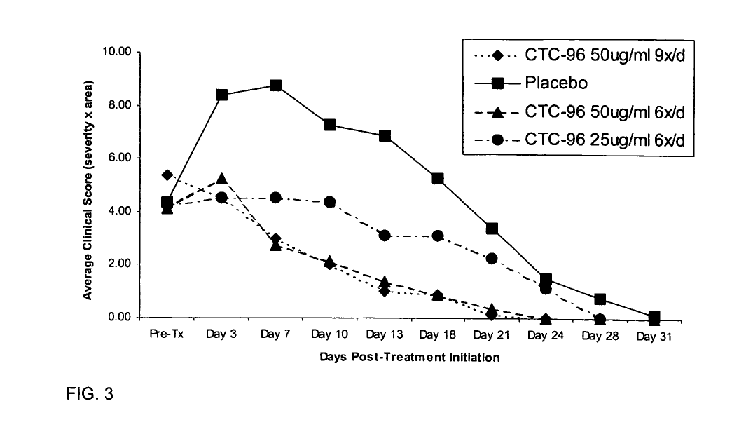

The results are depicted in FIG. 3 which shows CTC-96 treatment of Adenovirus induced keratoconjunctivitis. The data In FIG. 3 were obtained as follows: rabbits were infected with Human Adenovirus Type 5 by the installation 10⁶ pfu adenovirus according to our protocol of conjunctival and corneal scarification for the production of Keratoconjunctivitis. On day 8 post-inoculation treatment with eye drops containing CTC-96 or placebo was initiated. Animals were examined for stromal keratitis and scored by the corneal disease scale of Wander et al. (5). The following are the Criteria For Determination Of Conjunctival Disease:

| | Area of Conjunctival | | |
|---|---|---|---|
| | Disease | | Conjunctival Severity |
| 0 | Normal cornea. | 0 | Normal conjunctiva. |
| +1 | ≦25% involved. | +1 | Mild conjunctival injection. |
| +2 | >25%, ≦50% involved. | +2 | Moderate conjunctival injection/ |
| | | | chemosis. |
| +3 | >50%, ≦75% involved. | +3 | Severe conjunctival injection/ |
| | | | chemosis. |
| +4 | >75%, ≦100% involved. | +4 | Pseudomembrane present. |

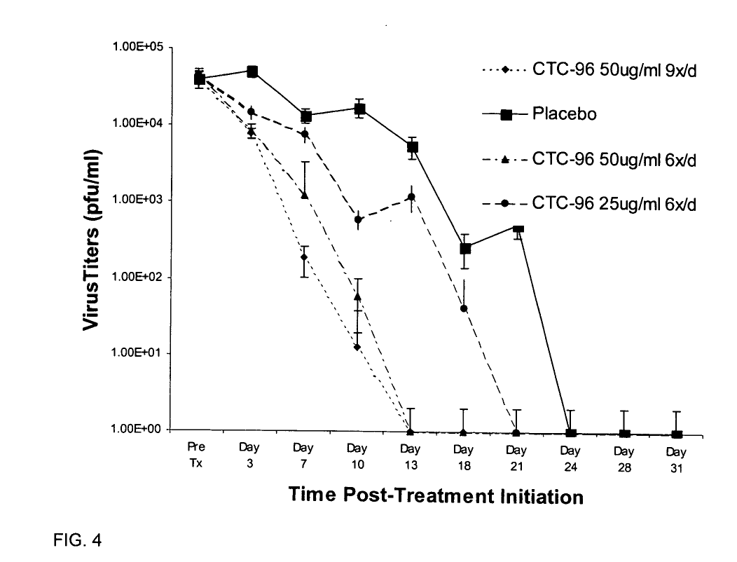

The efficacy of CTC-96 treatment of rabbit eyes infected with Human adenovirus, Adenovirus type 5 (Ad5), was also evaluated by adenovirus recovery from tear film cultures adsorbed onto confluent HeLa cell monolayers. Application of 50 μg/ml 6 or 9 times a day resulted in a rapid fall in viral presence in the eye with no detectable virus by day 13 while placebo treated eyes continued to show detectable virus until day 24. FIG. 4 shows adenovirus titers after treatment of rabbit eyes with CTC-96 or placebo. These data were obtained by the following procedure: rabbits were infected with Human Adenovirus Type 5 by the installation 10⁶ pfu adenovirus according to our protocol of conjunctival and corneal scarification for the production of Keratoconjunctivitis. On day 8 post-inoculation treatment with eye drops containing CTC-96 or placebo was initiated. Adenovirus recovery from tear film was evaluated by plaque assay on confluent HeLa cell monolayers. Data are presented as Average±SD.

1. A method for the therapeutic treatment of a subject infected by adenovirus comprising administering an anti-adenovirus-effective amount of CTC-96 to the subject.

2. The method of claim 1, wherein the adenovirus infection is an adenovirus-derived keratoconjunctivitis.

3. The method of claim 2, wherein the CTC-96 is administered topically.

4. The method of claim 1, wherein the adenovirus infection is a respiratory infection.

5. The method of claim 4, wherein the CTC-96 is administered nasally.

6. The method of claim 4, wherein the CTC-96 is administered by nasal spray or nose drops.

7. A method for the prophylactic treatment of a subject susceptible to infection by adenovirus comprising administering a prophylactic anti-adenovirus effective amount of CTC-96 to the subject.

8. The method of claim 7, wherein the infection to which the subject is susceptible is keratoconjunctivitis.

9. The method of claim 8, wherein the CTC-96 is administered topically.

10. The method of claim 7, wherein the infection to which the subject is susceptible is a respiratory infection.

11. The method of claim 10, wherein the CTC-96 is administered nasally.

12. The method of claim 10, wherein the CTC-96 is administered by nasal spray or nose drops.

13. A method for the therapeutic or prophylactic treatment of a subject infected by adenovirus comprising administering an anti-adenovirus-effective amount of CTC-96 to the subject wherein the disease is selected from the group consisting of: Acute febrile pharyngitis, Acute respiratory disease, Acute hemorrhagic cystitis, Epidemic keratoconjunctivitis, Gastroenteritis, Hepatitis, Meningoencephalitis, Pertussis-like syndrome, Pharyngoconjuctival fever, pediatric Pneumonia, and adult Pneumonia.
